(19) United States
(12) Patent Application Publication
(10) Pub. No.: US 2010/0010610 A1
(43) Pub. Date: Jan. 14, 2010

(54) ANASTOMOSIS STENT AND GRAFT APPARATUS AND METHOD

(75) Inventor: Mark Allen Grevious, Chicago, IL (US)

Correspondence Address: HUSCH BLACKWELL SANDERS LLP, 190 CARONDELET PLAZA, SUITE 600, ST. LOUIS, MO, 63105-3441, US

(73) Assignee: The Board of Trustees of the University of Illinois, Chicago, IL (US)

(21) Appl. No.: 11/908,470

(22) PCT Filed: Mar. 16, 2006
(86) PCT No.: PCT/US06/09601

§ 371 Date: Oct. 2, 2009

Related U.S. Application Data
(60) Provisional application No. 60/663,277, filed on Mar. 18, 2005.

(51) Int. Cl. A61F 2/06

(52) U.S. Cl. 623/1.11; 623/1.15; 623/1.13

(57) ABSTRACT

An anastomosis graft/stent apparatus comprising a first stent portion, a second stent portion and a graft. The graft is between the first and second stent portions. A port is in the graft portion and provides access to a vessel lumen at the anastomosis site, with the access being sufficient to remove thrombotic material.

### CROSS-REFERENCE TO RELATED APPLICATIONS

This application claims the benefit of U.S. Provisional Application No. 60/663,277, filed Mar. 18, 2005 and/or to international application PCT/US2006/009601 filed Mar. 16, 2006.

### BACKGROUND OF THE INVENTION

1. Field of the Invention

The present invention is in the field of stents and grafts as used in vascular surgery.

2. Related Art

Free tissue transfer is a medical technique used to address the frequently devastating loss of tissue suffered by patients who have been treated surgically for tumor resections, trauma or other ailments. In this technique, a portion of tissue is surgically freed from a donor site elsewhere on the patient's body and grafted onto the injury site in order to close the wound and provide cosmetic amelioration of the cosmetic consequences of the original surgery. Typically, the portion of tissue transferred is large enough to require a substantial amount of vascularization. This is done by carefully removing a site corresponding to the vascular tree of a particular donor vessel, and isolating the main donor vessel so that it is free and exposed for reattachment with a corresponding recipient vessel that has been freed and exposed at the recipient site of the wound.

Since the donor tissue will not have any collateral vascularization, as do many other tissues in the body, its survival is entirely dependent on the anastomosis of the donor vessel end with the recipient vessel end. There are a variety of known complications that can compromise the juncture of the donor and recipient vessels including rupture, vessel collapse, and thrombosis or clotting. Any of these events can reduce or halt blood flow to the donor tissue, causing it to become hypoxic, and then necrotic. In the event of necrosis, the tissue transfer has failed and the donor tissue must be removed.

A variety of mishaps can compromise the patent lumen necessary for a successful anastomosis. These include failure of the donor vessel end and the recipient vessel end to match. A mismatch may be due to a failure of the two vessel ends to conveniently reach each other for anastomosis after the donated tissue has been placed. A mismatch can also be caused by a non-parallel orientation of the two vessels during grafting, which can cause a kink in the vessels at the anastomosis. Problems also arise with mismatched diameters of the donor and recipient vessels. Iatrogenic anastomosis failures can be caused by accidental suturing of the back and near walls of the vessel together. This potential is exacerbated by the frequently small size of the vessels in question, which can be as small as 1 millimeter in diameter and smaller. Another concern for a successful anastomosis is matching intima at the anastomosis. The intima is the smooth inner lining of each vessel, which can be fragile, but which is necessary for the smooth laminar flow of blood cells through the vessel. In the event of an intima tear, or mismatched suturing of the vessels that fails to join the intima, the blood has a tendency to clot at the site and cause a thrombosis. There is a need in the art for equipment to help assure a successful anastomosis during free tissue transfer procedures.

Grafts and stents are known in the medical arts. Most known grafts are designed for axial placement. That is, in for example cardiac procedures, a stent is placed on the end of a catheter. Then the catheter enters the patient's body at a remote site and is passed, usually under fluoroscopic guidance, to the site of the pathology being treated. The stent is deposited there through any of a variety of release mechanisms, after which the catheter is withdrawn. These systems are impractical for free tissue transfer anastomoses, which are performed during open surgery when the surgeon has a lateral access to the anastomosis being placed, not an axial access.

Grafts are also known and used in some vascular surgeries, such as aneurysm repair or vascular access procedures for AV Graft dialysis patients. These grafts are made out of known materials. Placement of them typically involves a lateral access to a large pathologic vessel, such as the aorta, opening that vessel, placing the graft inside, suturing it in place and closing the vessel over it. This type of graft is also impractical for free tissue transfer since the vessels in question are too small for such an approach. The creation of longitudinal cuts on donor and recipient vessels during free tissue transfer is impractical for a number of reasons, including an increased complication rate.

There is a need in the art for a stent and/or graft apparatus to facilitate anastomoses, particularly in free tissue transfer procedures. As with all vascular surgery, there is a continuing need to reduce the amount of time needed for surgical procedures in order to reduce the time that the tissue downstream of the surgical site is without blood flow and oxygen.

There is a continuing need in the art to prevent post surgical thrombosis. After a free tissue transfer has been performed, it is important to monitor blood flow across the anastomosis in order to insure its continued patency. Moreover there is a need in the art for equipment to facilitate treatment of the anastomosis site should complications arise. Such treatment may include removal of clotted blood cells in the event of a thrombosis and may also include the administration of appropriate pharmaceuticals, such as thrombolytics.

Stents are constructed to have a preconfigured diameter and amount of structural rigidity in order to support the vessel in which they are deployed. The most successful technique for placement of stents is to mechanically or otherwise compress the stent radially so that it has a small diameter before placement and then mechanically or otherwise expand the diameter of the stent to the desired dimension after it is in position. There is a need in the art for an anastomosis stent that can be installed in a first diameter and expand to a second preconfigured diameter after placement wherein the apparatus for expansion accommodates lateral removal, rather than axial removal of the compression apparatus.

### BRIEF DESCRIPTION OF THE DRAWINGS

The present invention will become more fully understood from the detailed description and the accompanying drawings, wherein:

FIG. 1a is a perspective view of the stent graft apparatus of the present invention.

FIG. 1b is a side view of the stent graft apparatus of the present invention.

FIG. 2 is a side view of a second version.

FIG. 3 is a side view of another embodiment of the present invention.

FIG. 4 is a side view of another embodiment of the present invention.

FIG. 5 is a side view of another embodiment of the present invention.

FIG. 6 is a side view of another embodiment of the present invention.

FIG. 7a is a side view of a wire installation device, unexpanded.

FIG. 7b is a side view of a wire installation device, partially expanded.

FIG. 8a is a side view of a balloon installation device, unexpanded.

FIG. 8b is a side view of a balloon installation device, partially expanded.

FIG. 8c is a side view of a balloon installation device, partially installed.

FIG. 9 is a side view of the invention with a flow monitor.

FIG. 10 is a pre-curved embodiment.

FIG. 11 is a side view of an interposition vascular graft.

FIG. 12 is an interposition vascular graft having vascular couplers.

FIG. 13 is a side view of a stent graft apparatus showing one embodiment of a retention wire that can be laterally withdrawn.

FIG. 14 is a side view of the stent graft apparatus with a gel port.

FIG. 15 is a view of a graft stent apparatus showing a locking port.

FIG. 16 is an end view of another embodiment of a graft stent apparatus with a retractable retention wire in a compressed position.

FIG. 17 is an end view of another embodiment of a graft stent apparatus with a retractable retention wire in an expanded position.

FIG. 18 is a cut-away perspective view of another embodiment of a graft stent apparatus with a retractable retention wire in a compressed position.

FIG. 19 is a two wire version of the retention loops.

FIG. 20 is a single wire version of the retention loops.

FIG. 21 is a vascular coupler.

### SUMMARY OF THE INVENTION

The present invention is a graft/stent apparatus for use in an anastomosis. The apparatus is constructed and arranged to facilitate a lateral placement approach.

The present invention has either a full length stent or two end portion stents. The stents have a first diameter to be maintained before and during placement, and a second diameter after placement. Expansion may be achieved mechanically, either through insertion, expansion, decompression and withdrawal of a balloon, or through withdrawal of a wire. In either case, withdrawal of a mechanical actuator is from a lateral site in the graft/stent apparatus.

The apparatus of the present invention includes a port. Through this port, the patency of the anastomosis may be ascertained. Further, through this port thrombotic material may be removed. Further, through this port, medicinal pharmaceuticals can be administered.

The graft/stent of the present invention has a center portion incorporating graft material. This material may receive and maintain sutures for connection of vessel ends to the graft. In one embodiment of the invention, an annular collar is provided for receiving sutures. In another embodiment of the present invention, a vessel coupler is provided.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION OF THE PREFERRED EMBODIMENTS

The following description of the preferred embodiment(s) is merely exemplary in nature and is in no way intended to limit the invention, its application, or uses.

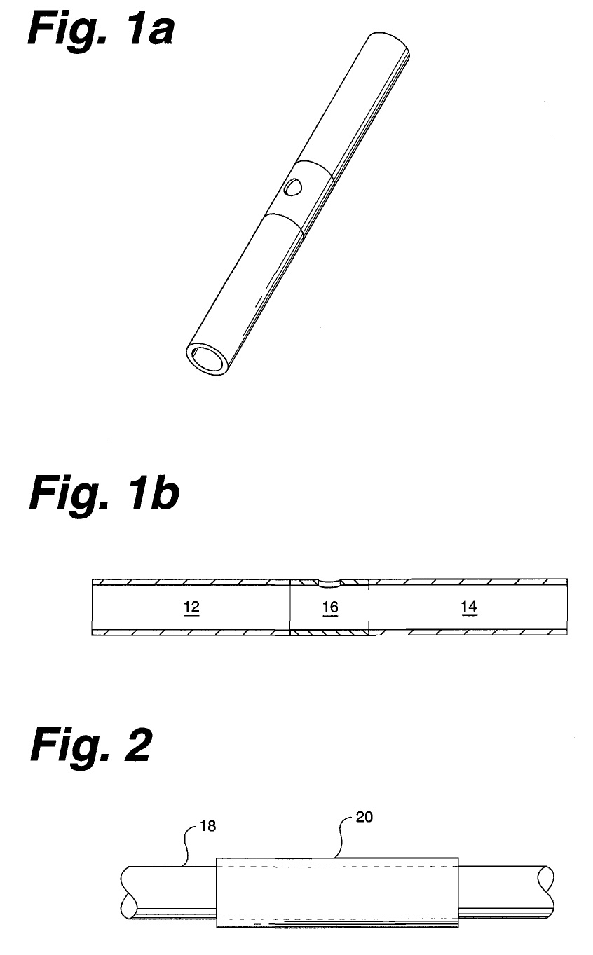

Referring now to the figures wherein like reference numbers indicate like elements, FIG. 1 depicts the graft/stent combination of the present invention. The apparatus 10 includes a first end comprised of a first stent 12 and a second end comprised of a second stent 14. A center portion of the apparatus is comprised of graft material 16. Graft material may be made of any appropriate material, including expanded PTFE, sometimes known as Gortex®.

FIG. 2 depicts another embodiment of the present invention. In FIG. 2 stent 18 runs the entire length of the device. Graft material 20 is superimposed circumferentially around and on the outside of the stent 18. In its deployed, expanded position, the inner surface of graft material 20 is substantially in complete contact with the outer surface of stent 18. Accordingly, as in the embodiment of FIG. 1, the first and second ends of the stent 18 in their expanded position extend into the ends of the donor and recipient vessel and support them in their preconfigured diameter against collapse, kinking and the like. In both configurations graft material 16, 20 is available for suturing to the ends of the vessels.

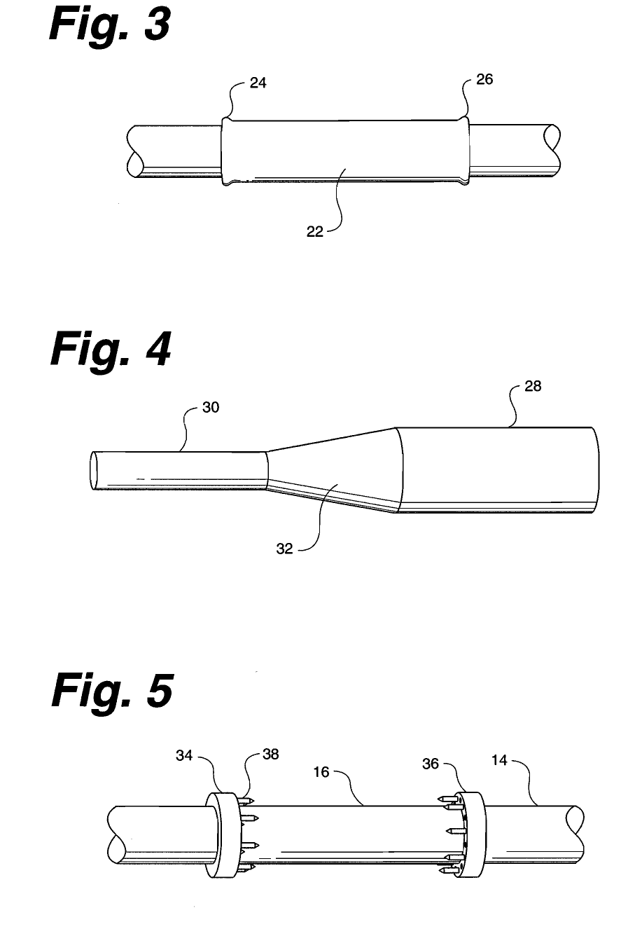

Another challenge faced by surgeons is a less than perfect match between the end of the donor vessel and the end of the recipient vessel. One of the variety of mismatches that can occur is when the ends of the two vessels do not conveniently reach each other for suturing. The Gortex® graft 16, 20 is available for suturing to the end of either or both vessels in order to bridge any gap between them. The end of a first vessel is sutured directly to the graft in a first position and the end of the other vessel is sutured to the graft at a second position remote from the first position along the axis of the device. FIG. 3 depicts an embodiment of the present invention specifically designed to accommodate such a circumstance. Annular collars 24, 26 are provided at either end of the graft material 22. The collars may be fabricated by attaching the grafting material 22 to the stent at a position inward of the end of the graft material 22 such that a fringe of the graft material 22 forms the annular collar 26, and leaves those collars available for suturing to the end of a vessel. Alternatively, the graft material, which is typically a molded or extruded polymer, such as PTFE, can be molded or extruded in order to have a thicker component at the ends of the tube and these thicker components would comprise collars 24, 26.

FIG. 11 depicts another embodiment of the present invention. In the version depicted in FIG. 11 the expanded PTFE 122 graft is as long as or longer than the stent 118, so that annular collars 126 extend beyond the ends of both. Annular collars 126 also extend radially beyond the diameter of graft 122 in order that they may provide material for the surgeon to suture to the ends of the respective donor and recipient vessels. Hence, in use, the embodiment of FIG. 11 will provide an interposition wherein the end of the recipient vessel would be attached to one end of the graft/stent apparatus at one annular ring 126 and the end of the donor vessel would be attached to the other annular collar 126 at the opposite end of the graft stent apparatus. Thus, the gap between the ends of the two vessels is bridged by the graft stent apparatus and the end of the recipient vessel and donor vessel never actually come into contact with each other. An alternative application for this construction would be vascular access procedures for AV Graft dialysis patients.

Another possible mismatch between the vessel ends appears when a first vessel has a first diameter and the other vessel has a different diameter. The apparatus of the present invention may be constructed and arranged to accommodate some variation in the diameters of the vessels being joined. FIG. 4 depicts a first stent 30 and a second stent 28 having different diameters. They are joined by a section of graft material 32 that is frustoconical 32.

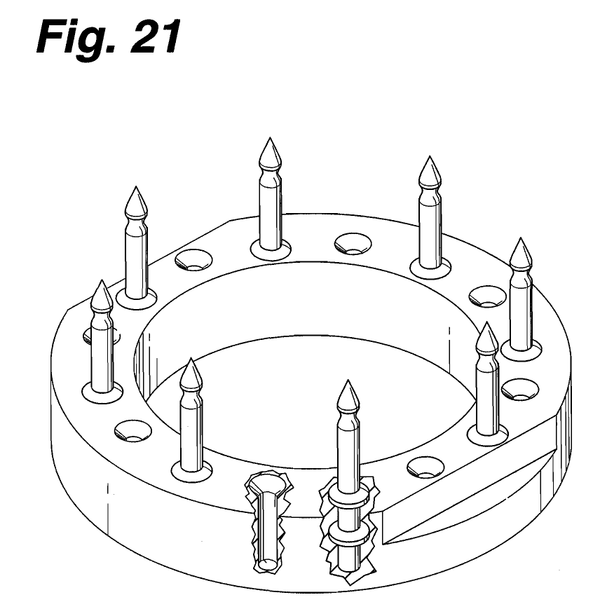

Recently a useful coupler for facilitating anastomoses has become available in the field. It consists of a first and second annular ring, each corresponding to the end of one vessel. One such annular coupler is depicted in FIG. 21 (prior art). The vessels, anastomosis and ring are coaxial. Each ring has on it a series of holes and pins running longitudinally parallel to the axis of the vessels and anastomosis. The pins are dimensioned to closely cooperate with the holes of the opposing ring so that the two coupling rings may be coupled together in a snap fit to form a single solid ring. In order to create an anastomosis a first vessel end is put through a first ring and the vessel end is expanded radially to approximate the mating surface of the coupler. The pins are used to pierce the expanded vessel wall and hold it in place. The same procedure is followed on the opposing vessel end. Thereafter the coupler is snapped together, securing the vessel ends proximate to one another. In this way the coupler achieves the goal of mating vascular intima and thereby providing a smooth surface to promote laminar flow of the blood through the anastomosis. Use of the coupler is especially advantageous in vein anastomoses.

This coupling arrangement may be incorporated into the stent/graft device of the present invention. A single coupler would be placed anywhere along the length of the stent or graft, preferably over the graft. In the embodiment depicted in FIG. 5, the graft 16 and stent 14 have placed over the complimentary and opposed rings 34 and 36. Each of rings 34 and 36 have pins 38 oriented to correspond to holes in the opposite ring. The holes alternate with the pins in each ring and are disposed to receive the pins of the opposing ring for closing the anastomosis and to closely cooperate with the holes of the opposing ring, as for example by a snap fit, in order to hold the anastomosis. In operation, a surgeon would place a first coupler over a first vessel end, the second coupler over the second vessel end, place a first stent end inside the first vessel and a second stent end inside in the second vessel and then join the vessels with the coupler to create an anastomosis over the stent/graft assembly.

Couplers may also be useful in the situation where the end of the donated vessel fails to reach the end of the recipient vessel. In such a case two couplers may be used, as depicted in FIG. 12. In the embodiment of FIG. 12, the stent 114 is surrounded by the graft 116 and at each end of the combined apparatus is a coupler half, 136A and 134B. One coupler is used at each end of the interposition graft/stent apparatus to attach one of the vessels ends to each end of the graft/stent apparatus. So for example, the surgeon would put a first coupler half 134A of the first coupler over the donor vessel and, as before, the end of that vessel would be expanded radially outward to be pierced by the pins 138 of the first coupler half 134A. In that position the first coupler first half 134A would be snapped into the first coupler second half 136A at one end of the graft/stent apparatus. Likewise, the second coupler second half 136B would be installed over the recipient vessel and the end of the recipient vessel pinned with pins 138 as before. Then the second half 136B of the second coupler would be snapped into place with the first half 134B of the second coupler, thereby firmly attaching the end of the recipient vessel to the other end of the graft/stent interposition apparatus. Thereby, using the interposition graft/stent apparatus combined with end couplers, a lumen patent for blood flow is created without the end of the recipient vessel and the end of the donor vessel ever touching each other.

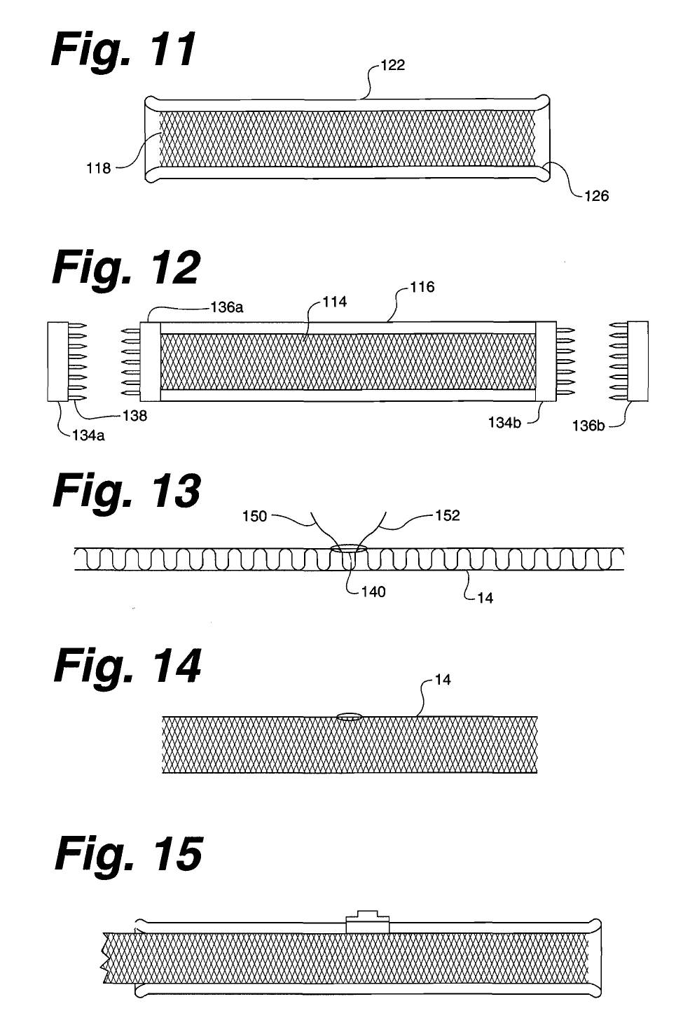

FIG. 10 depicts an embodiment of the present invention that is precurved. Another discrepancy between the ends of donor and recipient vessels is finding that they are non-parallel in their final placement position. Attempting to bend live vessel tissues to achieve a curve often results in kinking at the anastomosis. A precurved stent avoids this problem.

The structure of the graft/stent apparatus helps prevent misplacement of sutures, which is often difficult in microsurgery. The graft/stent apparatus may also be advantageously used with robotic surgical aids for minimally invasive surgery techniques or microsurgery. One such device is the DaVinci® robot which has small grasping instruments that may be controlled by a surgeon at a haptic interface under video guidance. The surgeon's control station may be nearby the surgical site or remote. Site access is important in such microsurgery.

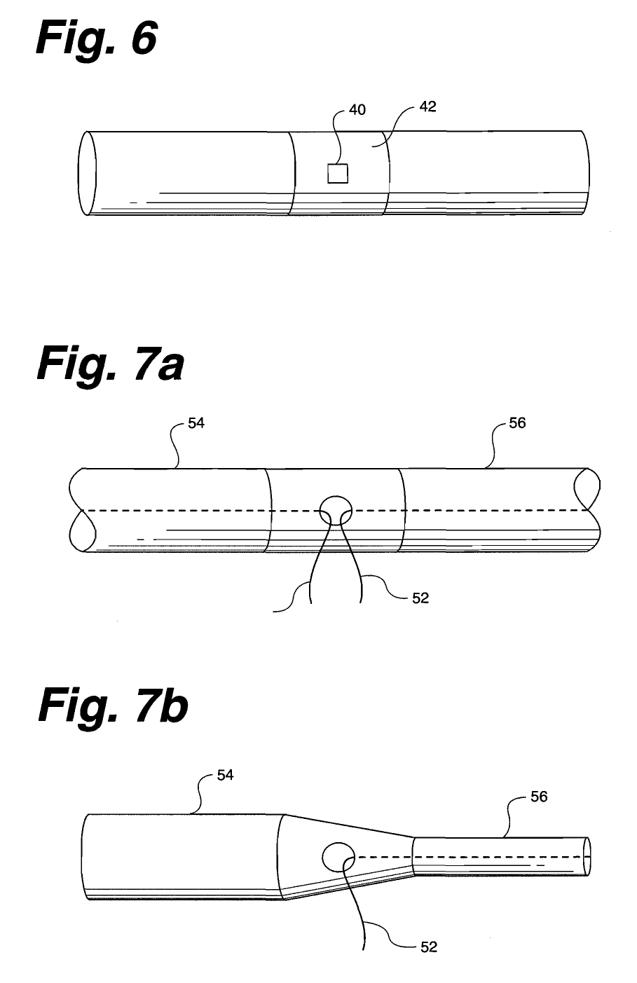

Another novel feature of the graft/stent device of the present invention is a port. FIG. 6 depicts the port 40 located along the wall of the graft 42. Port 40 is multifunctional. Port 40 may be used as an access point for delivery of medication. Port 40 would be positioned under an anastomosis, or between sutured junctures of first and second vessel ends to a graft material 42. In either case, the port may be traversed for the delivery of appropriate medication, such as thrombolytics. A second possible function of port 40 is an access to the lumen of the vessel in order to either first observe a thrombosis forming or secondly remove a thrombosis that has already formed.

A third function of port 40 or an alternate port is to allow mechanical withdrawal of an expansion device. Another novel feature of the present invention is the ability to install the stent portion of the device in a compressed position and deploy it within a vessel in an expanded position and to do so at an anastomosis site by center withdrawal of the expansion device. In the depicted embodiment, the expansion device is mechanical. In the embodiment depicted in FIGS. 7A and 7B a wire is deployed axially through each end of the stent before installation. Wire 50 and 52 are engaged with the stent ends 54 and 56 in order to retain them in a relatively narrow diameter, compressed first position. Upon installation within the vessel, a first wire 50 may be withdrawn thereby releasing the mechanical engagement of wire 50 with stent portion 54 and in turn allowing stent portion 54 to expand to its preconfigured final diameter within the vessel. FIG. 7A shows both stent ends 54 and 56 in their first, compressed position with the wires 50 and 52 remaining in place. FIG. 7B depicts a first stent end 54 in its expanded, installed second position after wire 50 has been withdrawn. FIG. 7B shows the opposing stent end 56 still in its compressed position, before wire 52 has been withdrawn.

The majority of stents have an interrupted and flexible structure that can be expanded and contracted in a radial direction, and that is inherently biased towards a rest diameter that is the expanded diameter. Usually it is biased towards an expanded position that approximates the desired diameter of the vessel in which it is used. Frequently they are installed by compressing them mechanically, as for example with a sleeve, and then removing the mechanical compression to allow them to expand to the rest diameter towards which they are inherently biased. The materials of such structure may be wire, plastic or other materials. They are frequently sinusoidal in a circumferential direction, with axially sequential sinusoidal rings either proceeding in a spiral, or as a series of conjoined rings. Examples of this type of structure appear in U.S. Pat. No. 6,312,459 B1 to Huang, see FIGS. 3 through 9, U.S. Pat. No. 5,967,986 to Cimochowski et al., see FIGS. 11 and 22, and U.S. Pat. No. 6,736,842 B2 to Healy et al., see FIGS. 1 and 4. In the embodiment depicted in FIG. 7 and FIGS. 16 through 20, any such structure, including without limitation the examples given in the prior art patents, that has a compressed narrow diameter and an uncompressed wider diameter towards which it is naturally biased may be used.

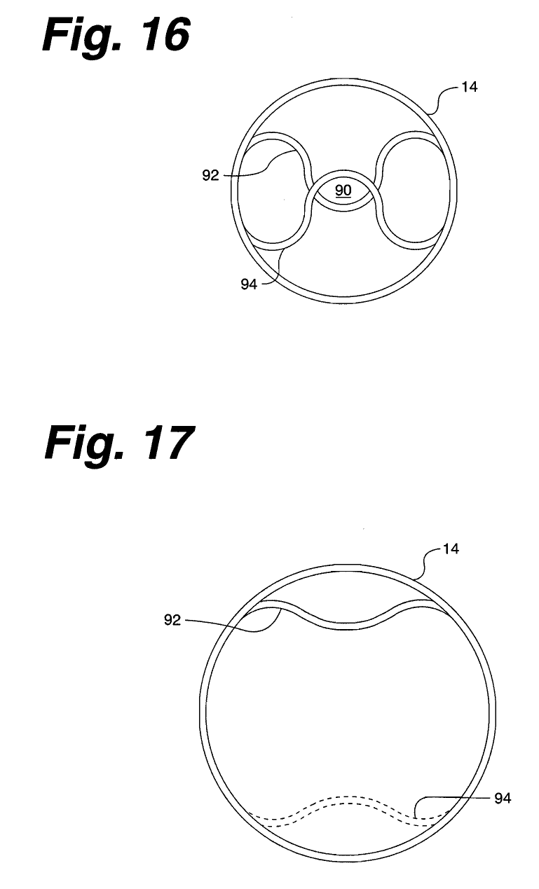

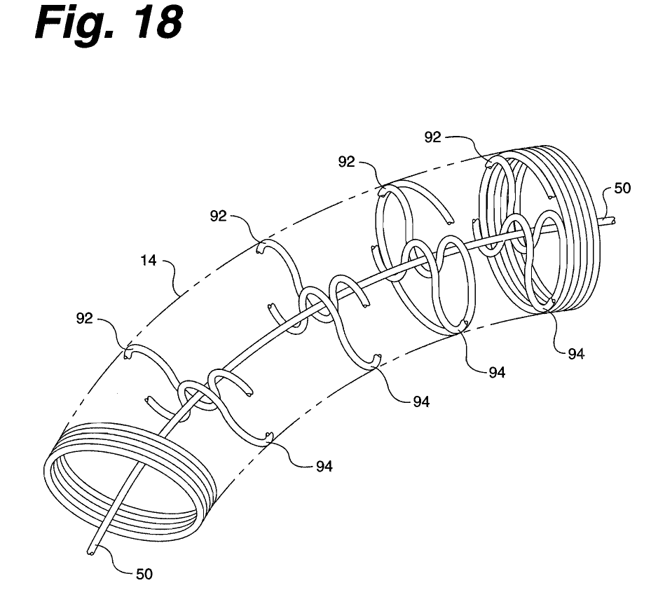

In FIGS. 16 through 20 are depicted a mechanical apparatus for achieving compression from within the lumen of the stent. The internal retention loops 90 and 94 depicted in FIGS. 16 through 20 may be advantageously used with the laterally retractable retention wires 50 and 52, disclosed in FIG. 7. FIG. 16 is an end view of a stent 14 in which periodic retention loops 92 and 94 are included. FIG. 16 depicts the stent portion of the apparatus in its compressed form. As can be seen, adjacent top loop 92 and bottom loop 94 overlap to create space 90. It is through space 90 that one of the retention wires 50 or 52 proceeds axially. FIG. 17 depicts the stent portion of the apparatus in its expanded position, after the retention wire is withdrawn. The diameter in FIG. 17 would be larger than that depicted in FIG. 16. As can be seen, retention loops 92 and 94 have retracted from their initial overlapping position to an expanded position in which they do not overlap. FIG. 18 is a cut away perspective view showing a stent 14 including a series of top retention loops 92 approximately paired with opposing retention loops 94. The retractable retention wire 50 is inserted through all of the loops serially. As depicted in end view 16, retractable retention wire 50 would proceed through space 90 created between the overlapping retention loops 92 and 94. By retracting the removable retention wire 50, the loops are released into their expanded position. In their contracted position, the loops are naturally biased radially outwards such that loops 92 and 94 will create opposing forces on retention wire 50. They would accordingly then be caught from complete expansion and retained in a compressed position by contact with retaining wire 50.

Retaining loops 92 and 94 may be comprised of any material, as for example metal wire. They are structurally joined with the other material and structure comprising the expanded stent 14. They may be integrally formed with the stent material and structure or formed separately and attached. They may be comprised of periodic loops in a spiral wire, sinusoidal or otherwise, or they may be periodically attached loops in a series of axially attached rings. The spacing of retention loops 92 and 94 may be in any advantageous dimension, both in relation to the opposing retention loop and in relation to the next axially sequential retaining loop. Each successive loop may vary circumferentially from the preceding loop.

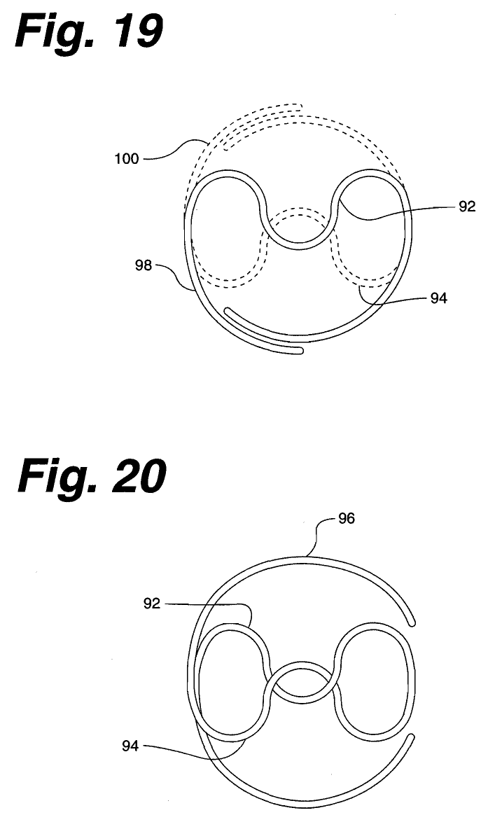

In FIG. 20 is depicted a single strand through 720 degrees of rotation such that the strand 96 forms both the upper retaining loop 92 and the lower retaining loop 94. In FIG. 19 two wires 98 and 100 may be used to configure the upper retaining loop 92 (created as a bend in wire strand 98 as it proceeds through 360 degrees) and the lower retaining loop 94 (created as a bend in wire strand 100 as it proceeds through 360 degrees). Again, the strands may be wire, or any other appropriate material.

FIG. 13 depicts an alternate configuration for retention of the stent in a compressed position that may be advantageously used with a lateral withdrawal. It depicts the stent 14 being held in place by a retention wires 150 and 152 that wrap in a spiral or helical fashion around the outside of the structure of the stent 14. Alternately, as the retention wire 150 may be interspersed or woven in and out of the structure of the stent material 114. Retention wire 150 proceeds laterally along the stent/graft apparatus in a first direction and retention wire 152 proceeds laterally along the stent/graft apparatus in the opposite direction. In operation, this device, like that depicted in FIG. 7, is installed in its compressed condition first. Then, after the anastomosis is complete, the retention wires 150 and 152 are withdrawn through the port 140. After the withdrawal of both wires, the stent material 14 expands to its integrally biased diameter as depicted in FIG. 14. This is the diameter for which the particular graft/stent apparatus was chosen by the surgeon to approximate the diameter of the donor and recipient vessels that the graft/stent apparatus is joining.

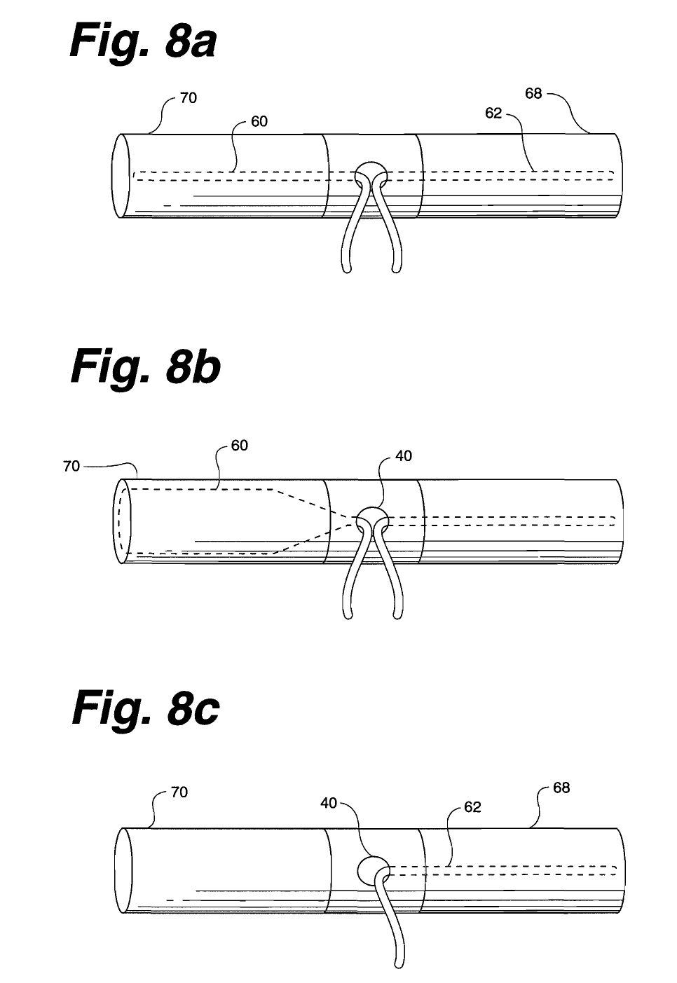

FIGS. 8a and 8b depict a second mechanical installation device, a balloon. Like wires, there is a first balloon 60 and a second balloon 62 are each deployed in an installation balloon catheter. In FIG. 8a both stent ends 68 and 70 are depicted in their compressed or first positions substantially circumscribing deflated balloons 60 and 62. In FIG. 8b a first stent end 70 is shown in its second, expanded position with the balloon 60 having been inflated in order to position the stent end 68 in the expanded position. After stent end 68 has been expanded to its deployed position it will remain in that position, thereupon balloon 60 may be deflated and withdrawn through port 40. FIG. 8C depicts a first stent end 68 in its final expanded, installed position after the withdrawal of deflated balloon 60.

A post-surgical concern in free tissue transfer cases is the continued patency of the lumen through the anastomosis in order to insure continuing blood flow to the transported and grafted tissue. It is anticipated that the structure of the graft/stent apparatus may advantageously be coated with antithrombitic medication such as paclitaxel or sirolimus. The graft material may be impregnated with antithrombotic microspheres or with antibiotic microspheres. Microspheres may be made up of different absorbable compounds such as polygalactin or hyaluronic acid.

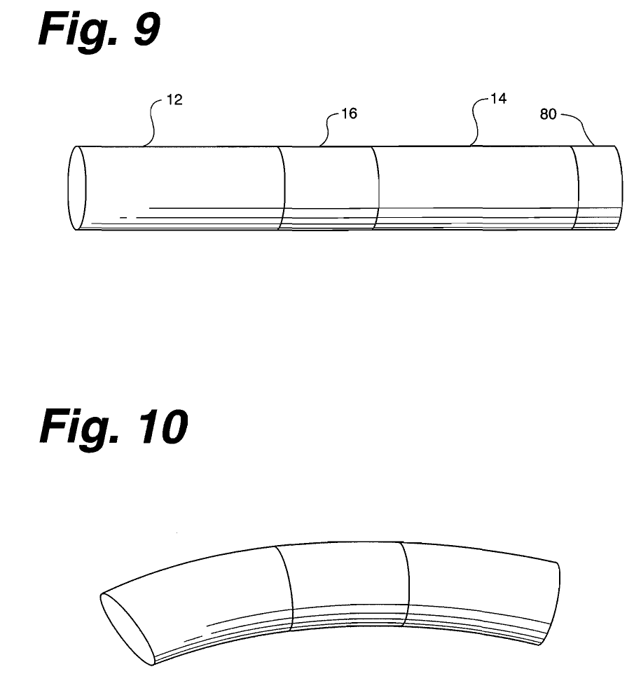

Devices exist to monitor blood flow, as for example by Doppler ultrasound. The sensor devices for such blood flow monitors can be quite small. FIG. 9 depicts a deployment of the stent graft device of the present invention in combination with insitu blood flow monitor. The blood flow monitor can be connected to a monitoring computer and display through wires (which may be cut after healing) or by radio frequency communication, which requires no wires. In the embodiment depicted in FIG. 9, blood flow monitor 80 is attached to one end of the stent or as a separate device that is slipped over one vessel before placement of the stent and retained there. The sensor device 80 may be placed at any position along the axial length of the graft/stent apparatus of the present invention.

FIGS. 13, 14 and 15 depict the port in greater detail. In FIGS. 13 and 14 a gel port 140 is depicted. The gel port has the capability of being pierced by a hard sharp object such as a hypodermic needle but, after such an object is withdrawn, reexpanding into the hole created the needle with sufficient elasticity and pressure to reseal the hole. One material known for use in such resealing gel ports is a silicon elastomer. One example of a silicon elastomer gel port put to a different use is a tissue expander port such as that used in the Mentor Corporation's Radovan™ Tissue Expander. Accordingly, such a gel port may be used for the insertion of appropriate medications and for the insertion of a needle of sufficient gauge to remove thrombolytic or clot material or a cannula sufficient for insertion of a wire, small scope or tube sufficient to inspect and if necessary mechanically remove problematic structures such as a thrombosis or clot.

Alternatively, as depicted in FIG. 15, the port 40 in the graft/stent apparatus may be a luer lock. The term “luer lock” is commonly known by those of skill in the medical arts as referring to one or more commonly used structures that have the operational capacity to temporarily attach an external device such as a syringe to an in site device such as an intravenous line, for example to add medication. The commonly known luer lock is threaded so that it is attached with a twisting motion. Typically, a male portion has at least one helical ramp on its outer surface that twists into a corresponding ramp, channel or boss on the inner aspect of a corresponding female portion. Close cooperation with the ramps may include a boss and detent configuration to achieve a snap fit when the turn of the operator's hand is complete and the device locks and seals into place. The term “lock” as used in this application shall mean any fixture allowing the attachment, removal, replacement and/or reattachment of an external hardware device with the graft/stent apparatus of the present invention at the lateral port. This will include but not be limited to luer locks, threaded locks, or snap fit locks. As is known with luer locks and is within the scope of the present invention with all locks, a seal facility may be included. That is, a mechanical flap, trap, plunger or the like can be activated by the locking application of the external device with the port lock. Alternately, a cap may be placed over the port upon removal of another external device. As with the gel port, any of these devices may be used to apply medicine, inspect the lumen within the graft/stent apparatus or allow access to the lumen in order to remove from it blockages such as a clot.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. An anastomosis graft/stent apparatus comprising:
a first stent portion;
a second stent portion;
a graft, said graft being between said first and second stent portions; and
a port, said port being in said graft portion and said port providing access to a vessel lumen at the anastomosis site, said access being sufficient to remove thrombotic material.

2. The apparatus of claim 1 wherein said port access is sufficient to deliver medication therethrough.

3. The apparatus of claim 1 further comprising at least one annular suture site.

4. The apparatus of claim 3 wherein said annual suture site is at an end of said graft.

5. The apparatus of claim 1 further comprising a coupler.

6. The apparatus of claim 1 wherein said stent portions each have a first compressed diameter and a second expanded diameter.

7. The apparatus of claim 1 wherein said first stent portion has an expanded diameter and said second stent portion has an expanded diameter and wherein said expanded diameters of said first and second stent portions are different.

8. The apparatus of claim 1 further comprising a flow meter.

9. The apparatus of claim 1 wherein said first and second stent portions are portions of a single continuous stent.

10. The apparatus of claim 6 wherein said first compressed diameter is maintained by an installation device.

11. The apparatus of claim 10 wherein said installation device is withdrawn from a substantially intermediate position along said graft/stent apparatus.

12. The apparatus of claim 10 wherein said installation device is withdrawn through a port in said graft.

13. The apparatus of claim 10 wherein said installation is mechanical.

14. The apparatus of claim 10 wherein said installation device is a wire, said wire being engaged with said stent portions to maintain said stent portions in said first compressed position until said wire is withdrawn.

15. The apparatus of claim 10 wherein said installation device is at least one balloon.

16. An anastomosis graft/stent apparatus comprising:
a first stent portion;
a second stent portion;
a graft, said graft being between said first and second stent portions and attached thereto;
said first and second stent portions each having a first compressed diameter maintained by an installation device and said first and second stent portions having a second, expanded diameter after installation;
an installation device, said installation device being engaged with a first and second stent portion to maintain them in said first compressed diameter until installation and said installation device being withdrawn from a position intermediate to a first end and a second end of said anastomosis graft/stent apparatus.

17. The apparatus of claim 16 further comprising a port, said port being intermediate to a first end and second end of said anastomosis graft/stent apparatus.

18. The apparatus of claim 17 wherein said port is sufficient to provide access for removal of thrombotic material.

19. The apparatus of claim 16 wherein said graft/stent apparatus is coated with antithrombitic medication.

20. The apparatus of claim 16 wherein said graft is impregnated with antithrombotic microspheres or with antibiotic microspheres.

21. The apparatus of claim 16 wherein said apparatus is robotically installed.

22. The apparatus of claim 16 wherein said apparatus is a vascular access AV Graft for dialysis patients.
